SEQUENCE LISTING (2 sequences)

SEQ ID NO: 1 - DNA, 25 bp, Artificial Sequence
  Artificially synthesized primer sequence
atcgatgataagctgtcaaacatga

SEQ ID NO: 2 - DNA, 17 bp, Artificial Sequence
  Artificially synthesized primer sequence
tcacagcattttttgct

(19) United States
(12) Patent Application Publication
(10) Pub. No.: US 2003/0170783 A1
(43) Pub. Date: Sep. 11, 2003

(54) Method to extend the life span of normal cells

(76) Inventor: Renu Wadhwa, Ibaraki (JP)

Correspondence Address: JANIS K. FRASER, PH.D., J.D., Fish & Richardson P.C., 225 Franklin Street, Boston, MA, 02110-2804, US

(21) Appl. No.: 10/107,555

(22) Filed: Mar. 26, 2002

Related U.S. Application Data
(63) Continuation-in-part of application No. PCT/JP00/06653, filed on Sep. 27, 2000.

(30) Foreign Application Priority Data
Sep. 27, 1999 (JP) 11/272778

(51) Int. Cl. C12P 21/02; C12N 5/08; C12N 5/06; C12N 15/85

(52) U.S. Cl. 435/69.1; 435/455; 435/325; 435/320.1; 435/366

(57) ABSTRACT

The present inventors discovered that the life span of human normal diploid pulmonary fibroblasts MRC-5 could be extended by intracellular expression of mot-2. The use of mot-2 enables the immortalization of cells and/or the extension of the cell life span without transformation. The method of the present invention is applicable to the establishment of normal liver cells for producing albumin, and such.

This application is a continuation-in-part of International Patent Application No. PCT/JP00/06653, filed Sep. 27, 2000, which claims priority to Japanese Patent Application No. 11/272778, filed Sep. 27, 1999.

### TECHNICAL FIELD

The present invention relates to a method that utilizes mortalin-2 protein to extend the life span of normal cells.

### BACKGROUND

Normal human cells divide a number of times until they reach termination of cell division, and ultimately enter a state, in terms of metabolism, where the growth of the cell is permanently inhibited (replicative senescence) (Hayflick et al., Exp. Cell Res. 25:585-621, 1961). Because of various mechanisms, including genetic and epigenetic alterations, cancer cells divide permanently under normal culture conditions, and can escape the limit of replication ability. The number of genes involved in this process and its nature are far from completely understood. Quite rarely, human cells get spontaneously immortalized. In addition, expression of a viral oncogenes can extend the life span of cells. Nonetheless in both the scenarios the cells enter a stage called “crisis” during which there is no net increase in cell number although a viable cell populations can be maintained through long periods. In most cases, extremely few cells (10⁻⁶ to 10⁻⁹) escape the crisis stage and become immortalized.

In recent years, the telomere hypothesis about life span regulation has gained recognition, due to the result of analyses on telomere length and telomerase activity in normal and cancer cells. All cancer cells can maintain their telomere length which otherwise erode with progressive population doublings in normal cell. Telomere damage in one or more chromosomes of normal somatic cells is conjectured to trigger cell senescence. Therefore, reactivation of the telomerase or other telomere maintenance pathways that function in place of telomerase is thought to be necessary for cell immortalization (Bryan et al., Nat. Med. 3, 1271-4, 1997; Bryan et al., EMBO J. 14:4240-8, 1995; Harley et al., Cancer Surv. 29:263-84, 1997; Reddel, Jpn. J. Cancer Res. 88:1240-1, 1997). On the other hand, there is clear evidence that cellular senescence involve mechanisms independent to that of telomere shortening. By microcell fusion approach introduction of specific chromosomes in aparticular cell line resulted in senescence phenotype and it was not always associated with telomerase inactivation. In fact, telomerase activity has been detected in many senescent cultured cells (Oshimura et al., Eur. J. Cancer 33:710-5, 1997). Recently, it was reported that embryo cells of Syrian hamster underwent senescence even though they retained the telomerase activity and the lengths of the telomeres do not decrease (Carman et al., Exp. Cell Res. 244:33-42, 1998). Expression of catalytic component of telomerase did not interfere with premature senescence of normal fibroblasts induced by the Ha-Ras oncogene (Wei et al., Cancer Res. 59:1539-1543, 1999). These findings supported the presence of a senescence mechanism(s) independent of telomere counting mechanism.

The role of p53 and pRb tumor suppressor genes in cell senescence has been elucidated by numerous independent studies (Bond et al., Oncogene 13:2097-2104, 1996; Gire et al., Mol. Cell Biol. 18:1611-21, 1998; Kulju et al., Exp. Cell Res. 217:336-45, 1995; Serrano et al., Cell 88:593-602, 1997; Vaziri et al., Exp. Gerontol. 31:295-301, 1996; von Zglinicki et al., Z. Gerontol. Geriatr. 30:24-8, 1997). It was shown that the late passage human fibroblasts have stronger DNA binding and p53 transcription activation ability of the passage (Atadja et al., Proc. Natl. Acad. Sci. USA 92:8348-52, 1995; Bond et al., (1996) Oncogene 13:2097-2104, 1996). Expression of mutant p53 in senescent human fibroblast and microinjection of p53 antibody into cells has been reported to induce cell division (Bond et al., Oncogene 9:1885-9, 1994; Gire et al., Mol. Cell Biol. 18:1611-21, 1998). Escape from senescence has been shown to be closely related to lack of p53 function(Gire et al., Mol. Cell Biol. 18:1611-21, 1998; Harvey et al., Genes Dev. 5:2375-85, 1991; Levine, Cell 88:323-31, 1997; Wynford-Thomas, J. Pathol. 180:118-21, 1996; Wynford-Thomas, Eur. J. Cancer 33:716-26, 1997; Wynford-Thomas et al., Biol. Signals 5:139-53, 1996). Recently, it was shown that cell cycle arrest due to p19^{ARF} is mediated by wild-type p53 (Chin et al., Trends Biochem. Sci. 23:291-6, 1998; Sherr, Genes Dev. 12:2984-91, 1998), and p21^{WAF-1} (Kamijo et al., Proc. Natl. Acad. Sci. USA 95:8292-7, 1998; Palmero et al., Nature 395:125-6, 1998; Zhang et al., Proc. Natl. Acad. Sci. USA 95:2429-34, 1998). p21^{WAF-1} is a cyclin-cdk inhibitor that is at least partially regulated by p53. The role of p21^{WAF-1} in cell senescence has been verified by the up-regulation of p21^{WAF-1} in senescent fibroblasts and by extension of life span due to disruption of p21^{WAF-1} in normal diploid cells (Brown et al., Science 277:831-4, 1997; El-Deiry et al., Cell 75:817-825, 1993; Noda et al., Exp. Cell Res. 211:90-8, 1994).

Expression of many other genes has been characterized in relation to normal and transformed phenotype cells (Berube et al., Am. J. Hum. Genet. 62:1015-9, 1998; Chang, J. Formos. Med. Assoc. 96:784-91, 1997; Duncan et al., Biochemistry (Mosc.) 62:1263-74, 1997; Haber, Cell 91:555-8, 1997; Kaul et al., Ind. J. Exp. Biol. 36:345-352, 1998; von Zglinicki et al., Z. Gerontol. Geriatr. 30:24-8, 1997).

Mortalin was the first gene that was cloned by its relation to the division phenotype of cells The present inventors reported that mortalin is distributed homogeneously in the cytoplasm of normal cells, and is localized around the cell nucleus in immortalized cells (Wadhwa et al., J. Biol. Chem. 268:6615-21, 1993; Wadhwa et al., Exp. Cell Res. 207:442-8, 1993).

Recently, the present inventors demonstrated that inactivation of normal p53 transcription activity is induced by mot-2, but not by mot-1. Physical interaction between mot-2 and p53 was detected, as was the inhibition of p53 nuclear translocation and location of p53 to the cytoplasm by the mot-2 protein (Wadhwa et al., J. Biol. Chem. 273:29586-91, 1998). In addition, the present inventors discovered that over-expression of mot-2 causes malignant transformation of NIH3T3 (Kaul et al., Oncogene 17, 907-11, 1998), and that the transformation may be enhanced by the inactivation of the function of normal p53 (Wadhwa et al., J. Biol. Chem. 273:29586-91, 1998). Furthermore, the inactivation of p53 by mot-2 may lead to maintenance of unphosphorylated pRB by p53-p21-pRB network (Weinberg, Cell 85:457-9, 1996). Such decrease in p21^{WAF1} expression by mortalin (Wadhwa et al., J. Biol. Chem. 273:29586-91, 1998) may interfere with the cellular senescence phenotype of normal cells.

Malignant transformation of nontumorigenic immortalized cells, such as NIH3T3 cells (immortal cells that are not tumorigenic but can be cultured endlessly and do not have a limited cell life span), has been reported as the effect of overexpression of mot-2 in these cells. However, the functional impact of mot-2 on normal cells, which undergo senescence (cells that have limited proliferative life span), has not been reported until now.

Primary culture cell lines of normal cells are technologically extremely important for the analysis of cell function, but normal cells cannot be cultured for a long period due to cell senescence. Furthermore, the cell function often cannot be maintained for a long period. Thus, limitations exist in the technical use of primary cells. Therefore, immortalized cells transformed by viruses and chemical mutagens have been technologically used as cultured cell lines.

However, cell culture systems that maintain their physiological functions are required for analysis of particular types of cell function, for production of useful substances. Therefore, techniques for long-term cultivation of cells and techniques to establish immortalized cells without using transformation have been desired.

### SUMMARY

The object of the present invention is to provide a method for extending the life span of normal cells. More specifically, the object of this invention is to provide a method for delaying death of normal cells due to senescence by elevating the level of mot-2 within the cells.

To investigate the effect of mot-2 on normal cells, the present inventors introduced mortalin-2 cDNA, which encodes a cytoplasmic protein that is localized around the nucleus (non-pancytoplasmic) and is isolated from immortalized mouse and human cells, into human normal diploid pulmonary fibroblasts MRC-5 at 31 PDs (population doublings). Then, the subsequent behavior of these cells was analyzed. The results in the control (a cell transfected with an empty vector) revealed that the growth of the cells became moderate after 28 PDs and no proliferation could be observed during any time in the next 35 days. These cells proved to be positive in the β-Gal staining, which is an index for senescence. On the other hand, the mot-2 gene transfected cells exhibited young morphology even after 28 PDs, and extended rounds of cell division by 12 to 18 PDs was observed. Decrease in β-Gal staining was also observed in these cells. According to these facts, the-present iventors concluded that the life span of MRC-5 cells could be extended by intracellular overexpression of mot-2.

In addition, the present inventors investigated the activity of p53 in MRC-5 cells wherein mot-2 is expressed, and, as a result, found that p53 inactivation was related to the extension of the life span of the MRC-5 cells. Furthermore, it was discovered that the extension of the life span of MRC-5 cells is positively correlated with the mot-2 protein expression level, determined by analysis of intracellular mot-2 protein level in MRC-5 cells that were confirmed to have extended life span.

Accordingly, the present invention provides a method for regulating the life span of normal cells by utilizing mot-2 protein, and more specifically provides the following:

(1) a DNA encoding mortalin-2 protein, which is used for extending the life span of normal cells;

(2) the DNA of (1), wherein the normal cells are human normal diploid pulmonary fibroblasts;

(3) a vector into which the DNA of (1) or (2) is inserted;

(4) a normal cell harboring the vector of (3);

(5) a method for extending the life span of a normal cell, comprising the step of elevating the intracellular mortalin-2 protein level;

(6) the method of (5), wherein the elevation of intracellular mortalin-2 protein level is caused by the introduction of a DNA encoding mortalin-2 protein into the cell; and

(7) the method of (5) or (6), wherein the normal cell is a human normal diploid pulmonary fibroblast.

This invention provides a method for extending the life span of normal cells by elevating the intracellular mot-2 protein levels. Herein, the term “normal cell” refers to a cell that has a limited life span and does not have tumorigenicity. Examples of such normal cells include human derived cells, such as TIG, HFF, WI-38, and human normal diploid pulmonary fibroblast MRC-5; however, the present invention is not limited to these examples. Elevation of the level of the mot-2 protein within these cells extends the life span of these cells and the cells will undergo extended population doublings (PDs).

No particular limitation exists on the type of mot-2 protein to be expressed at elevated levels. For example, known mot-2 proteins, such as mouse mot-2, human mot-2A, and human mot-2B, may be used in the present invention (Kaul et al., Oncogene 17:907-11, 1998). Methods for elevating the intracellular level of theses protein include, for example, a method wherein an expression vector expressing a mot-2 protein is introduced into the cell. No particular limitation exists on the expression vector, so long as it is an expression vector designed for animal cells; it can be exemplified by expression vectors that carry the SRα promoter or the CMV promoter. Alternatively, cells may be treated with chemical substances for elevating the expression of mot-2. 2-deoxy-glucoside can be exemplified as such a chemical substance, but the present invention is not limited thereto.

The method to extend the cell life span of this invention may be used, for example, to establish human cell lines with a known genetic background, by combining the method with the use of oncogenes and telomerases that induce immortalization of other cells. Such immortalized cells have immeasurable industrial utility.

For example, the extension of the cell life span of this invention may be used in human cells to establish normal liver cells for long-term production of albumin under culture conditions. Albumin production in liver cells has a great industrial value. Liver cells express albumin but could not be cultivated for extended periods without transformation, until now. Thus, in the past, it was difficult to obtain cultivated liver cells having similar morphologiy. The present invention enables long-term cultivation using mot-2 protein, and preparation of immortalized cells. This opens up new paths for the development of albumin production systems, the construction of biological evaluation systems that use liver cells, and the development of artificial organs.

### DESCRIPTION OF DRAWINGS

FIG. 1 is a set of photographs showing the results of mot-2 mRNA and protein detection. Panel A: mot cDNAs within MRC-5 cells, transfected either with the vector alone (the empty vector or control), or with the mot-2 expression vector, were detected by RT-PCR using vector primers and mortalin primers. RT-PCR, which was performed as a control experiment, was carried out using GAPDH primers. The mot cDNA was detected only in cells transfected with mot-2, hmot-2A, and hmot-2B. Panel B: the results of Western blot carried out using anti-mortalin antibody on MRC-5 cells that were transfected with the control vector and mot-2. As compared to untransfected cells and cells transfected with the vector alone, high levels of protein expression were detected in the cells transfected with mot-2, hmot-2A,-and hmot-2B gene.

FIG. 2 is a set of photographs showing the morphology of cells transfected with the control vector or hmot-2B gene in a subculture of MRC-5 cells. MRC-5/hmot-2B cells indicated younger morphology at 24 PDs compared to MRC-5/pSRα cells (comparison of panel b and f). MRC-5/hmot-2B cells at 40 to 46 PDs were similar to MRC-5/pSRα cells at 26 to 28 PDs (comparison of panel g and h to c and d).

FIG. 3 is a set of photographs that depict senescence dependent β-Gal staining of MRC/pSRαcells, MRC-5/hmot-2A cells, and MRC-5/hmot-2B cells. In the same generation of population doubling, β-Gal staining levels of hmot-2A cells and hmot-2B cells were observed to be lower as compared to those of the cells transfected with the control vector.

FIG. 4 is a graph depicting the number of population doublings (PDs) of MRC-5 cells transfected with the vector (pSRα), mot-2 cDNA (pSRα/mot-2), hmot-2A cDNA (pSRα/hmot-2A), and hmot-2B cDNA (pSRα/hmot-2B) under the culture condition. Cells at 31 PDs were transfected, then vector, mot-2, hmot-2A, and hmot-2B clones were subcultured in a G418 selection media up to 28, 37, 38, and 45 PDs, respectively. The total cell lifespan of the clones represented by population doublings were 59, 68, 69, and 76 PDs, respectively.

FIG. 5 is a set of graphs depicting the results of p53-dependent (A) or p53-independent (B) luciferase reporter assay. Control vector, mot-2, hmot-2A, and hmot-2B were transfected into MRC-5 cells at 21 PDs. The cells transfected with mot-2 showed five times lower p53-dependent activity as compared to those of the cells transfected with the control vector.

FIG. 6 is a set of photographs showing the results of a p53-dependent β-Gal reporter assay utilizing MRC-5 cells transfected with a control vector or hmot-2B at 25 PDs. Cells injected with a reporter plasmid were detected using a FITC-bound rabbit IgG secondary antibody. The number of blue cells among cells transfected with hmot-2B was significantly less compared to that among cells transfected with the vector.

### DETAILED DESCRIPTION

The present invention will be described in detail by way of Examples below; however, the present invention is not limited thereto. Human normal diploid pulmonary fibroblasts “MRC-5” used in the present invention were cultivated in DMEM medium (GIBCO) containing 10% fetal bovine serum. Cells were subcultured in a ratio of 1:4.

### Example 1

### Extension of the Life Span of MRC-5 Cells

cDNAs of mouse mot-2, human mot-2A (hmot2-A), and human mot-2B (hmot2-B3) were isolated from mouse MEF cell cDNA library, human fibroblastic tumor HT1080 cDNA library, and HeLa cells (human uterine cancer cells), respectively, by screening with mortalin antibodies.

These cDNAs, that contain the entire open reading frame of mouse and human mot-2 proteins, were cloned into a pSRα expression vector comprising the human immunodeficiency virus promoter/enhancer and the neo gene, and were transfected into MRC-5 cells at 31 PDs. Transfection was performed using LipofectAMINE™ (GIBCO). Transfectants were selected in DMEM medium containing 10% FBS, supplemented with 50 μg/ml of G418. Stabilized clonal cells were isolated by the Ring's method. Approximately 200 clonal cells were transferred to a 3.5-cm dish, and were cultivated until confluent. Then, the cells were subcultured in a 6-cm dish in a ratio of 1:4 to measure the cell life span (described below).

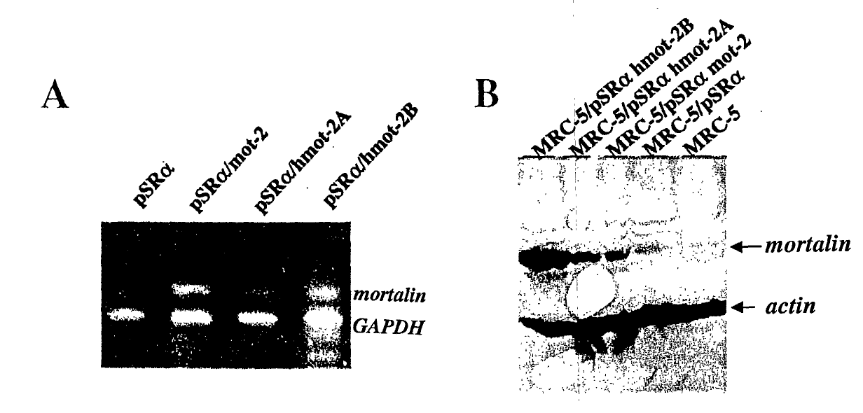

Expression of the mortalin gene due to exogenous cDNA introduced into the cloned cell was confirmed by RT-PCR. Specifically, first, total RNA was prepared from cells using TRIzol (GIBCO). RT-PCR was conducted on 1 μg of the total RNA, using a vector primer and mortalin primer. Sense primer, “5′-atc gat gat aag ctg tca aac atg a-3′ (SEQ ID NO: 1)”, and antisense primer, “5′-tca cag cat ttt ttg ct-3′ (SEQ ID NO: 2)”, were used. As a control, RT-PCR using GAPDH primer was performed. A DNA fragment of approximately 500 bp was detected by the RT-PCR (FIG. 1A). The nucleotide sequence of the amplified DNA product was determined, and the product was confirmed to be mortalin.

Two to three positive clones were selected from each of the transfected cells, and were cultivated to an amount sufficient for further analysis. SDS-PAGE was performed on protein solutions (10 μg each) prepared from cells transfected with the control vector alone and from cells transfected with the mot-2 gene. Then, the proteins on the gel were transferred to a nitrocellulose membrane (BA85, Schleicher and Schuell) using a semi-dry transfer blotter (Biometra, Tokyo). Anti-mortalin antibody or actin antibody (Boehringer Mannheim) was used in the immunoassays. Immunocomplexes were detected by ECL Kit (Amersham) using anti-rabbit IgG antibody covalently bound with horseradish peroxidase (Amersham). Consequently, excessive expression of mot-2 protein was confirmed in mot-2 gene transfected cells (FIG. 1B). The expression level of hmot-2B was higher compared to those of hmot-2A or mouse mot-2.

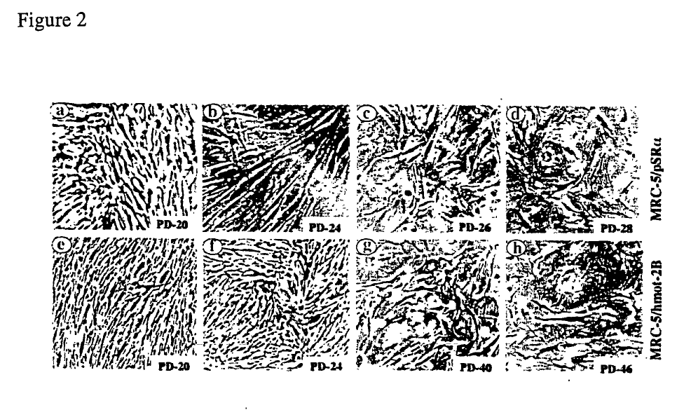

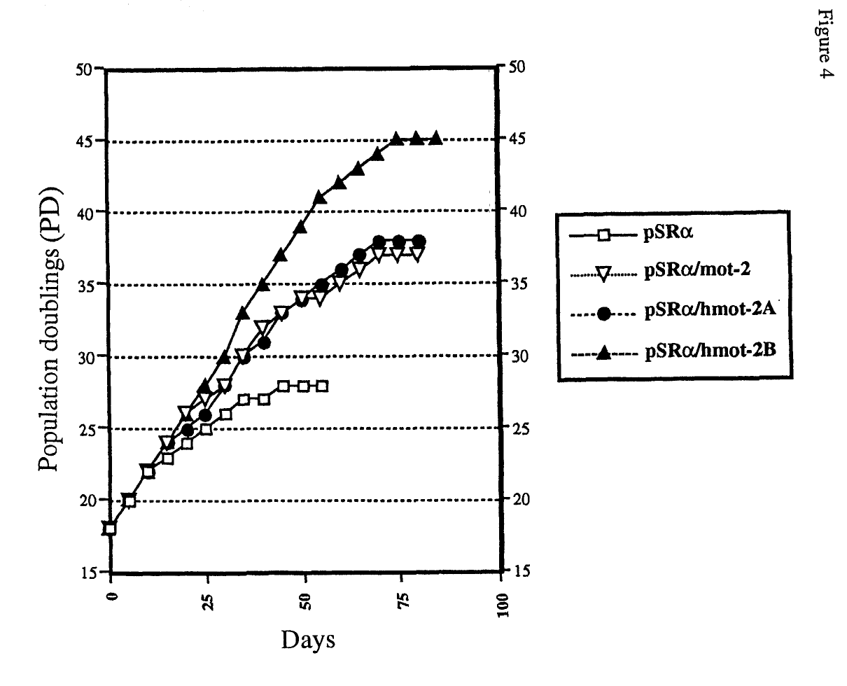

Expression vectors containing a cDNA encoding either mouse or human mot-2 protein were transfected into MRC-5 cells. Untransfected MRC-5 cells, MRC-5 cells transfected with the control vector alone (e.g., the empty vector), and MRC-5 cells transfected with the mot-2 gene were subcultured in a 6-cm dish in a ratio of 1:4. Subculturing was continued for at least 3 weeks, until marked increase in the number of cells could not be observed any more and cell division stopped. Population doublings(PDs) were calculated from the passage number until the cultivation could be continued. In untransfected cells and in cells transfected with the vector alone, senesced morphology was detected and cell death occurred after 28 PDs (FIG. 2). As compared to the control cells, cells transfected with mot-2 gene indicated young morphology up to 24 PDs. Morphology of these cells at 40 PDs was similar to the control cells transfected with the vector alone at 26 PDs (FIG. 2). In contrast to the fact that the control cells could only divide up to 28 PDs, the cells transfected with mouse mot-2, hmot-2A, and hmot-2B gene were subcultured up to 37, 38, and 45 PDs, respectively. These results demonstrated that the cell life span of MRC-5 at 9, 10, and 17 PDs was extended due to transfection of mot-2, hmot-2A, and hmot-2B gene, respectively (FIG. 4). According to the second experiment, the cell life span of cells transfected with the control vector was 30 PDs, while the cell life span of cells transfected with mot-2, hmot-2A, and hmot-2B were 44, 46, and 48 PDs, respectively. The results of these two experiments revealed that the cell life span of cells transfected with mot-2 gene was extended by 12 to 18 PDs from the point of the division termination in the cells transfected with the control vector gene.

The cells transfected with the control vector or the mot-2 gene were stained by substrate coloring utilizing endogenous β-galactosidase activity, which is an index of senescent culture cells. β-galactosidase staining was performed according to the procedures described in the literature (Dimri et al., Proc. Natl. Acad. Sci. USA 92:9363-7, 1995). The cells were washed with PBS at pH 7.2, and were fixed in PBS solution by the treatment with 2% formaldehyde/2% glutaraldehyde, or 4% formaldehyde for 10 minutes. Then, the fixed cells were colored at 37° C. in a freshly prepared solution containing 1 mg/ml 5-bromo-4-chloro-3-indolyl-β-D-galactoside, 40 mM citric acid-sodium phosphate (pH 6.0), 5 mM potassium ferricyanide, 5mM potassium ferrocyanide, 150 mM NaCl, and 2 mM MgCl₂.

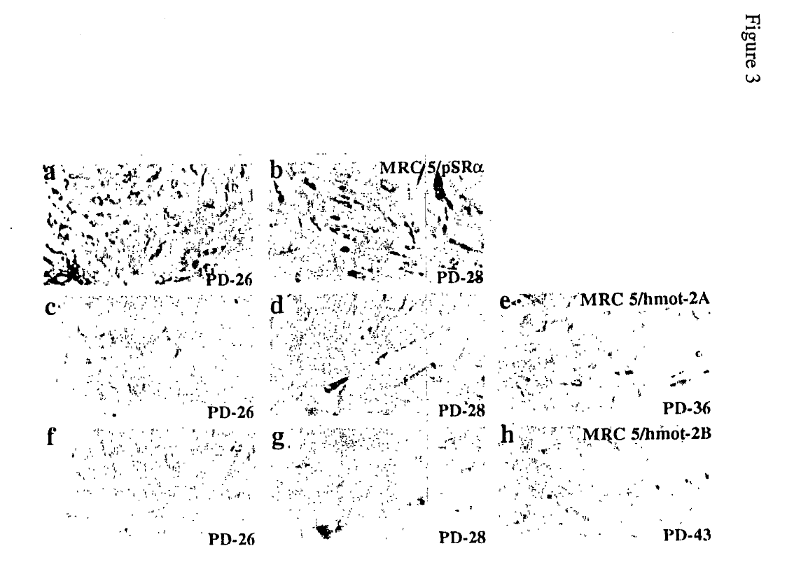

The cells transfected with the vector were stained with β-Gal at 26 and 28 PDs (FIG. 3a and b). However, at similar PDs, the number of cells that was stained blue had remarkably diminished in the mot-2, hmot-2A (FIG. 3c and d), and hmot-2B transfected cells (FIG. 3f and g). Increase in the number of β-Gal stained cells was observed among hmot-2A transfected cells of 28 to 36 PDs, and hmot-2B transfected cells of 28 to 43 PDs. However, at all PDs, cells transfected with mot-2 gene showed weaker staining as compared to cells transfected with the control vector.

### Example 2

### Cells with Extended Cell Life Span Showed Inactivation of p53 Transcription Activity

To investigate the p53 activity, p53 responsive luciferase reporter plasmid was transfected into cells, transfected with either the control vector or mot-2 gene at 21 to 25 PDs.

A p53 responsive luciferase reporter plasmid, pWWP-luc (containing a p21^{WAF1} promoter) (gift from Dr. Bert Vogelstein), was introduced into the cells by transfection. The gene transfection efficiency was measured by the co-transfection of pRL-CMV. Luciferase assay was performed 48 hours after the transfection using Dual-Luciferase™ Reporter Assay System (Gibco BRL). The amount of luciferase was calculated per 1 μg of protein and determined by the Bradford protein assay.

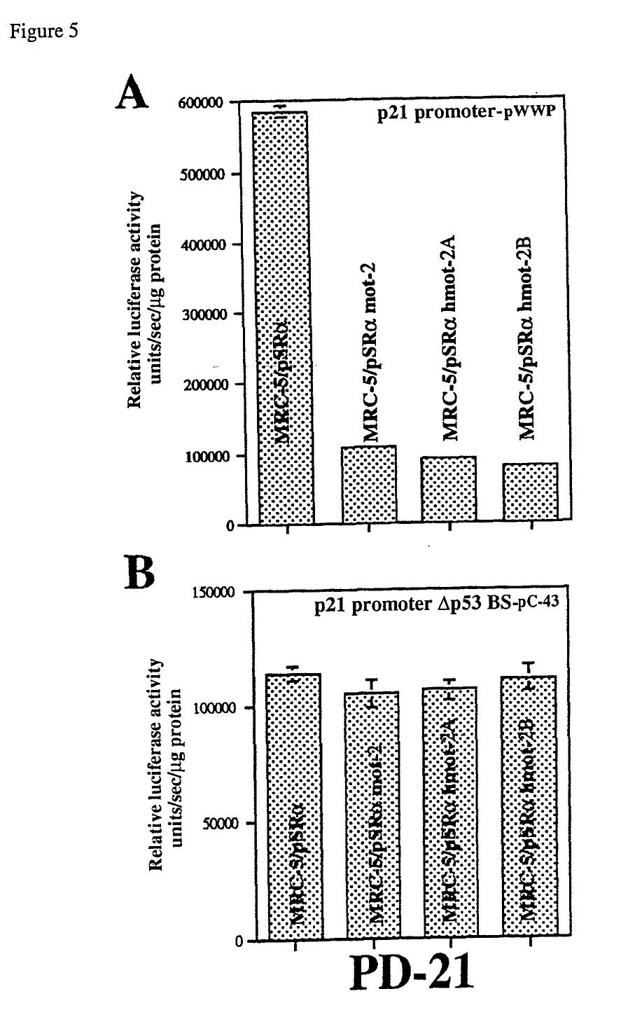

At 21 PDs, p53-dependent luciferase activity in the cells transfected with the control vector was 5 times higher than those of cells transfected with mot-2, hmot-2A, or hmot-2B gene (FIG. 5A). Additionally, measurements were also carried out using a mutant reporter plasmid lacking both of the two p53 binding sites. The cells transfected with the control vector or mot-2 vector demonstrated activities equivalent to p53-independent activity (FIG. 5B).

The transfection efficiency of normal cells at 26 PDs was extremely low. Consequently, the present inventors attempted to carry out microinjection of p53-responsive β-Gal reporter plasmid into cells transfected with mot 2 or the control vector, so that their cell life span would become the same.

Microinjection was conducted by a direct injection of p53-responsive β-Gal reporter, pRGC-fos-lacZ (having 13 repeating p53 binding sequences) (provided by Dr. David Wynford-Thomas), into the nucleus of cells proliferating on a cover slip, by utilizing Eppendorf semi-automated microinjection system (Eppendorf) mounted on an inverted microscope (Zeiss). The control IgG was co-injected as an index for the injected cells. After incubating the cells overnight, the cells were fixed in 4% formaldehyde for 10 minutes at room temperature and were washed with PBS. Permeabilization treatment was carried out on ice for 5 minutes in PBS containing 0.1% Triton X-100, and the treated cells were washed 3 times with PBS. The injected IgG was detected with secondary antibodies covalently bound to FITC, and the expression of β-galactosidase was detected using a β-Gal staining kit (Boehringer Mannheim). The cells were observed with a Zeiss microscope. Cells were counted as expression positive cells even if they were only stained faintly blue.

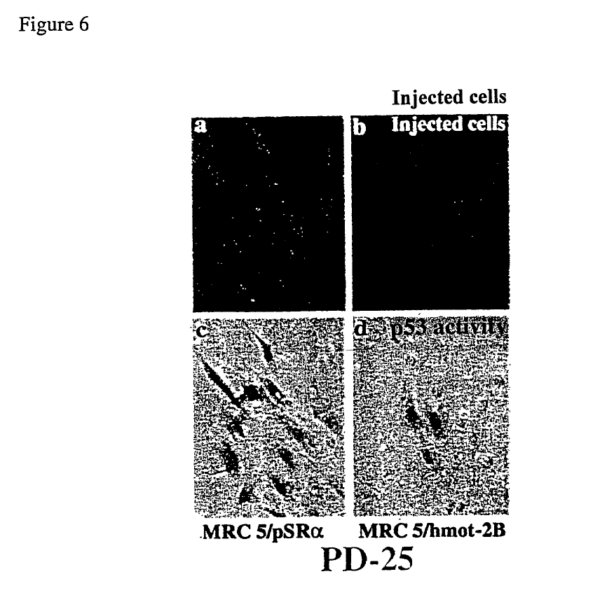

Approximately 200 to 250 cells were subjected to microinjection to measure ,β-Gal expression in two independent experiments. p53-responsive β-Gal coloring could be observed in 90 to 95% of the untransfected cells or those transfected with the control vector, while only 8 to 10% of the cells transfected with mot-2 gene were stained blue (FIG. 6). Similarly, only a weak coloration could be observed in young cells treated by microinjection (data not shown). These data indicate that p53 is activated in senescence, but its expression is considerably suppressed in cells transfected with mot-2 gene that have extended cell life span. Therefore, extension of cell life span may be caused by the level of p53 inactivation (inhibition of nuclear translocation) as described in an early report (Wadhwa et al., J. Biol. Chem. 273:29586-91, 1998).

These Examples indicate that mot-2 extends the cell life span of normal cells, and that mot-2 is involved, at least in part, in inactivating the p53 tumor suppressor factor. Mortalin-2 protein, DNA encoding the protein, and vectors including the DNA may serve as medicaments for extending the life span of normal cells.

### Industrial Applicability

The present invention provides a method for extending the cell life span of normal cells by utilizing mortalin-2. The method of this invention may be applied to establish human cell lines with known genetic background. Such immortalized cells have immeasurable industrial utility.

In addition, the extension of the cell life span according to the present invention may be useful with regard to human cells for establishing normal liver cells that produce albumin under culture conditions over a long-term. The method is predicted to be applicable to the development of albumin production systems, the construction of a biological evaluation system using liver cells, and the development of artificial organs.

What is claimed is:

1. A method of increasing the life span of a cell that normally undergoes senescence, the method comprising the step of increasing the level of mortalin-2 in the cell.

2. The method of claim 1, wherein the step of increasing the level of mortalin-2 in the cell comprises introducing a mortalin-2 encoding DNA into the cell.

3. The method of claim 1, wherein the step of increasing the level of mortalin-2 in the cell comprises administering to the cell a compound that increases expression of an endogenous mortalin-2 gene.

4. The method of claim 3, wherein the compound is 2-deoxy-glucoside.

5. The method of claim 1, wherein the cell is a diploid cell.

6. The method of claim 5, wherein the diploid cell is a fibroblast.

7. The method of claim 6, wherein the fibroblast is a pulmonary fibroblast.

8. The method of claim 5, wherein the diploid cell is a liver cell.

9. The method of claim 1, wherein the cell is a human cell.

10. The method of claim 1, wherein the mortalin-2 is selected from the group consisting of: mouse mortalin-2 and human mortalin-2.

11. The method of claim 10, wherein the mortalin-2 is human mortalin 2A or human mortalin 2B.

12. A method of increasing the life span of a cell that normally undergoes senescence, the method comprising administering a mortalin-2 encoding nucleic acid to the cell.

13. The method of claim 12, wherein the cell is a diploid cell.

14. The method of claim 12, wherein the cell is a fibroblast.

15. The method of claim 14, wherein the fibroblast is a pulmonary fibroblast.

16. The method of claim 12, wherein the cell is a liver cell.

17. The method of claim 12, wherein the nucleic acid is provided in a vector.

18. The method of claim 12, wherein the mortalin-2 is selected from the group consisting of: mouse mortalin-2 and human mortalin-2.

19. The method of claim 18, wherein the mortalin-2 is human mortalin 2A or human mortalin 2B.

20. A method of producing albumin, the method comprising:
(a) obtaining a liver cell comprising an exogenous mortalin-2 encoding nucleic acid;
(b) allowing the liver cell to express an albumin; and
(c) collecting the albumin from the liver cell, thereby producing albumin.

21. The method of claim 20, wherein the liver cell is a human liver cell.

22. The method of claim 20, wherein the nucleic acid encodes mouse or human mortalin 2.

23. The method of claim 25, wherein the mortalin 2 is human mortalin 2A or mortalin 2B.

24. A method of producing albumin, the method comprising:
(a) administering to a liver cell a compound that increases expression or activity of endogenous mortalin-2;
(b) allowing the liver cell to express an albumin; and
(c) collecting the albumin from the liver cell, thereby producing albumin.

25. The method of claim 24, wherein the liver cell is a human liver cell.

26. The method of claim 24, wherein the compound is 2-deoxy-glucoside.
